(19) United States
(12) Patent Application Publication
(10) Pub. No.: US 2026/0295002 A1
(43) Pub. Date: Oct. 1, 2026

(54) GASTROINTESTINAL RETENTIVE FORMULATIONS OF ECHINOCANDINS

(71) Applicant: The Cleveland Clinic Foundation, Cleveland, OH (US)

(72) Inventor: Thaddeus STAPPENBECK, Cleveland, OH (US)

(21) Appl. No.: 19/474,839

(22) PCT Filed: Apr. 12, 2024
(86) PCT No.: PCT/US2024/024447

§ 371 Date: Oct. 13, 2025

Related U.S. Application Data
(60) Provisional application No. 63/495,904, filed on Apr. 13, 2023.

(51) Int. Cl. A61K 38/12; A61K 9/00; A61K 47/10; A61K 47/32; A61K 47/38

(52) U.S. Cl. CPC A61K 38/12; A61K 9/0065; A61K 47/10; A61K 47/32; A61K 47/38

(57) ABSTRACT

Provided herein are methods and compositions for delivering echinocandins (e.g., caspofungin) to a subject to increase retention time in the gastrointestinal tract (e.g., to promote a localized action in the intestinal region and reduced absorption of the echinocandin) after oral administration of the composition. In certain embodiments, the compositions comprise an echinocandin and a carrier agent. In some embodiments, the compositions are used to treat fungal infections and/or inflammatory bowel disease in a subject.

The present application claims priority to U.S. provisional application Ser. No. 63/495,904 filed Apr. 13, 2023, which is herein incorporated by reference in its entirety.

### FIELD

Provided herein are methods and compositions for delivering echinocandins (e.g., caspofungin) to a subject to increase retention time in the gastrointestinal tract (e.g., to promote a localized action in the intestinal region and reduced absorption of the echinocandin) after oral administration of the composition. In certain embodiments, the compositions comprise an echinocandin and a carrier agent. In some embodiments, the compositions are used to treat fungal infections and/or inflammatory bowel disease in a subject.

### BACKGROUND

A variety of fungal infections can occur in patients due to pathogenic Candida or Aspergillus fungus species. Examples of such fungal infections include candidemia, candidiasis (including esophageal infections, abdominal infections, pleural space infections and peritoneal infections), and invasive aspergillosis. Early antifungal agents typically attacked the inner cell membrane of the invasive fungus. These early agents had a variety of drawbacks, however, including toxic side effects, drug-drug interactions, variations in efficacy between patients, and fungal resistance.

A more recent family of antifungal agents is the echinocandins, which treat fungal infections through a different mechanism-inhibition of the enzyme that forms β-(1,3)-D-glucan, an essential component of the fungal outer cell wall. Since β-(1,3)-D-glucan does not occur naturally in the cell walls of mammals, the action of echinocandins is unlikely to be harmful to the cells of an infected patient. Due to the difference in their mechanism of action relative to earlier agents, echinocandins have not experienced wide resistance by target fungi. Caspofungin acetate (Caspofungin) was the first of the echinocandins to be approved in the U.S. for use as an antifungal agent. The full name for caspofungin acetate is reported as 1-[(4R,5S)-5-[(2-aminoethyl)amino]-N2-(10,12-dimethyl-1-oxotetradecyl)-4-hydroxy-L-ornithine]-5-[(3R)-3-hydroxy-L-ornithine]pneumocandin B0 diacetate (salt), and a representative chemical structure of caspofungin acetate is shown further below.

As caspofungin acetate has poor oral bioavailability, it typically has been provided to medical personnel as a lyophilized solid, which is then reconstituted before intravenous administration to a patient. In one example, a formulation of caspofungin acetate that is commercially available at present is sold under the CANCIDAS® trademark. CANCIDAS® for Injection (Merck & Co, Inc.; Whitehouse Station, N.J., USA) is currently available as a lyophilized powder. CANCIDAS® is available in vials containing either 54.6 mg or 75.6 mg of caspofungin acetate, in combination with sucrose and mannitol, and including acetic acid and sodium hydroxide as pH modifiers. CANCIDAS® is reconstituted for administration by combining the lyophilized powder with 10.8 milliliters (mL) of a reconstitution liquid (such as 0.9% sodium chloride), to provide a solution having a caspofungin acetate concentration of either 7 milligrams per milliliter (mg/mL) or 5 mg/mL. This reconstituted liquid typically is diluted with an infusion liquid prior to administration.

### SUMMARY

Provided herein are methods and compositions for delivering echinocandins (e.g., caspofungin) to a subject to increase retention time in the gastrointestinal tract (e.g., to promote a localized action in the intestinal region and reduced absorption of the echinocandin) after oral administration of the composition. In certain embodiments, the compositions comprise an echinocandin and a carrier agent. In some embodiments, the compositions are used to treat fungal infections and/or inflammatory bowel disease in a subject.

In some embodiments, provided herein are compositions comprising: an echinocandin, and b) a carrier agent, wherein said carrier agent comprises at least one of the following: i) cellulose, ii) an acrylic acid derivative polymer, iii) a thermoreversible polymer, and iv) a thermoreversible polymer mixed with at least one ion-dependent polymer.

In particular embodiments, provided herein are compositions comprising: a) an echinocandin, and b) a carrier agent, wherein said carrier agent comprises at least one of the following: i) methyl cellulose, wherein optionally said methyl cellulose comprises hydroxypropyl methyl cellulose (HPMC), ii) low molecular weight chitosan, optionally in acetic acid, iii) polyvinylpyrrolidone (PV), iv) Pluronic F-127, wherein said Pluronic F-127 is optionally present in said composition at about 15-17% or about 13-19% (e.g., about 13, 14, 15, 16, 17, 18, or 19%); v) Carbopol, wherein optionally said Carbopol is in combination with said HPMC, and vi) thermo-sensitive poloxamer (F127), optionally in combination with a pH-sensitive polyacrylic acid (PAA).

In certain embodiments, at least one of the following: i) said methyl cellulose is present in said composition at about 1-3% (e.g., about 1, 2, or 3%), ii) said HPMC comprises 80-120 cp HPMC or HPMC 615, and/or iii) said PVP is present in said composition at about 0.5%-2.0% (e.g., about 0.5 . . . 0.8 . . . 1.2 . . . 1.5 . . . 1.8 . . . or 2.0%).

In certain embodiments, provide herein are methods of treating a subject (e.g., human) with a fungal infection and/or inflammatory bowel disease comprising: administering any of the compositions described above or below to the subject, or providing to the subject such that they administer the composition to themselves.

In particular embodiments, the administering or the administer the composition to themselves, comprises oral or intracolonic administration. In further embodiments, the subject has a fungal infection selected from: candidemia, esophageal candidiasis, and aspergillosis. In some embodiments, the subject has Crohn's disease.

In certain embodiments, the administering or the administer the composition to themselves, comprises oral administration; and wherein the echinocandin exerts its fungicide effect locally in the gastrointestinal tract.

In particular embodiments, the compositions herein are mucoadhesive. In other embodiments, the echinocandin comprises caspofungin. In further embodiments, the echinocandin comprises micafungin and/or anidulafungin.

In some embodiments, the compositions further comprise: water. In additional embodiments, the composition is in the form of a gel. In additional embodiments, the composition is configured to form a gel in situ when ingested orally. In some embodiments, the carrier agent comprises a thermoreversible polymer (e.g., further comprising at least one ion-dependent polymer). In certain embodiments, the compositions are configured to be gastrointestinal retentive when taken orally.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the technology may be readily combined, without departing from the scope or spirit of the technology.

In addition, as used herein, the term “or” is an inclusive “or” operator and is equivalent to the term “and/or” unless the context clearly dictates otherwise. In addition, throughout the specification, the meaning of “a”, “an”, and “the” include plural references. The meaning of “in” includes “in” and “on.”

As used herein, the terms “subject” and “patient” refer to any animal, such as a mammal like a dog, cat, bird, livestock, and preferably a human (e.g., a human with a fungal infection or inflammatory bowel disease).

### DETAILED DESCRIPTION

Provided herein are methods and compositions for delivering echinocandins (e.g., caspofungin) to a subject to increase retention time in the gastrointestinal tract (e.g., to promote a localized action in the intestinal region and reduced absorption of the echinocandin) after oral administration of the composition. In certain embodiments, the compositions comprise an echinocandin and a carrier agent. In some embodiments, the compositions are used to treat fungal infections and/or inflammatory bowel disease in a subject.

In certain embodiments, the echinocandin comprises caspofungin, which is picture below:

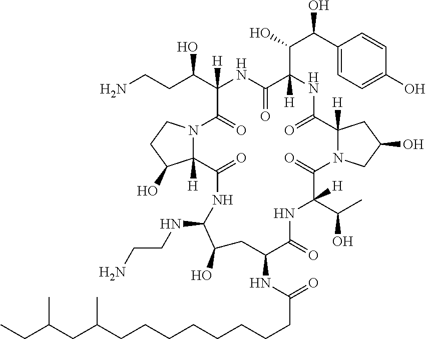 [SMILES: CCC(C)CC(C)CCCCCCCCC(=O)N[C@H]1C[C@@H](O)[C@@H](NCCN)NC(=O)[C@@H]2[C@@H](O)CCN2C(=O)[C@H]([C@H](O)CCN)NC(=O)[C@H]([C@H](O)[C@@H](O)c2ccc(O)cc2)NC(=O)[C@@H]2C[C@@H](O)CN2C(=O)[C@H]([C@@H](C)O)NC1=O]

Caspofungin, a lipopeptide currently administered intravenously due to the poor oral bioavailability, is employed inflammatory bowel disease indication such as Crohn's disease. Provided herein are formulations that allow for oral administration of caspofungin or other echinocandin. The formulations herein allow a echinocandin to exert the fungicide effect locally in the gastrointestinal tract. In certain embodiments, the compositions herein are able to increase the retention time of an echinocandin (e.g., caspofungin) in the gastrointestinal tract to promote a localized action in the intestinal region and reduced absorption of caspofungin. In certain embodiments, the compositions herein have a low viscosity (or tolerable viscosity for oral administration) before administration but able to form a gel once administered (e.g., gel formed in situ in a subject, such as a human).

### EXAMPLES

### Example 1

### Mucoadhesive Formulations Vehicle Testing

The mucoadhesive properties of certain formulations were investigated using a texture analyser. Adhesive tests were performed on the formulations containing 1% PVP k90 and 2% HPMC 615 as vehicles. Mucin tablets were prepared by compressing 300 mg of mucin powder (from porcine stomach, type II) with a manual hydraulic press (Graseby Specac®) using a 13 mm die applying a force of 5 tons for 60 seconds. The tablets were attached to the cylindrical probe (6 mm DIA CYLINDER STAINLESS) of the texture analyser with a double-sided adhesive tape. The samples were placed on the texture analyser plate and prior the adhesion tests, the mucin tablets were submerged in a 5% mucin suspension in 0.1 M HCl solution for 30 seconds. Afterwards, the probe was lowered at the speed of 1 mm/s until they come into contact, and the contact was held for 30 seconds applying a force equals to 10 g (0.1N). The probe was moved up at a constant speed of 2 mm/s. From the force vs distance plot, two parameters were determined the force maxima (Fmax), which is the maximum force required to separate the mucin discs to the samples and the work of adhesion (Wad), which is the amount of forces involved in the detachment. Higher force maxima was detected for formulation AP0546/8/01 compared to AP0546/7/01 as shown in Table 1.

TABLE 1

| | | Force maxima | Work of adhesion |
|---|---|---|---|
| ID | Vehicle | (Fmax), | (Wad), N · sec |
| AP0546/7/01 | 1% PVP k90 | 76.66 | 0.03 |
| AP0546/8/01 | 2% HPMC 615 | 166.86 | 0.02 |
| AP0546/17/01 | 2% HPMC 80-120 cP | 129.52 | 0.01 |

Additional testing was performed using 2% HPMC 80-120 cP (higher viscosity compared to HPMC 615) in order to obtain a better understanding how the viscosity is affecting the mucoadhesive properties of the vehicles. Minor differences were observed between the two HPMC vehicles, however, the higher viscosity vehicle is recommended in order to potentially increase the time that the adhesion is occurring.

Formulations were prepared using two additional vehicles (2% HPMC 80-120 cp and 0.5% chitosan LMW in 0.5% acetic acid) with higher viscosity known for their mucoadhesive properties (target concentration: 5 mg/g), as shown in Table 2.

TABLE 2

| | | | | | Theoretical | Concentration of |
|---|---|---|---|---|---|---|
| Formulation | | Visual | | | concentration | API dissolved in |
| ID | Vehicle | observations | pH | Assay (%) | (mg/g) | vehicle (mg/mL) |
| AP0546/18/01 | 2% HPMC 80-120 cp | clear solution | 6.33 | 98.1 | 4.9 | 4.8 |
| AP0546/18/02 | 0.5% chitosan LMW | clear solution | 4.06 | 98.4 | 4.9 | 4.8 |
| | in 0.5% acetic acid | | | | | |

API was Caspofungin. Ca. 98% assay was detected at t0 for both formulations. pH was higher for the formulation containing HPMC, potentially an additional analysis can be performed to confirm stability of samples (formulations were stored at 2-8° C., in closed glass vials).

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the technology as claimed should not be unduly limited to such specific embodiments. Indeed, various modifications of the described modes for carrying out the technology that are obvious to those skilled in pharmacology, biochemistry, medical science, or related fields are intended to be within the scope of the following claims.

We claim:

1. A composition comprising:
a) an echinocandin, and
b) a carrier agent, wherein said carrier agent comprises at least one of the following:
i) methyl cellulose, wherein optionally said methyl cellulose comprises hydroxypropyl methyl cellulose (HPMC),
ii) low molecular weight chitosan, optionally in acetic acid,
iii) polyvinylpyrrolidone (PV),
iv) Pluronic F-127, wherein said Pluronic F-127 is optionally present in said composition at about 15-17% or about 13-19%;
v) Carbopol, wherein optionally said Carbopol is in combination with said HPMC; and
vi) thermo-sensitive poloxamer (F127), optionally in combination with a pH-sensitive polyacrylic acid (PAA).

2. The composition of claim 1, wherein said echinocandin comprises caspofungin.

3. The composition of claim 1, wherein said echinocandin comprises micafungin and/or anidulafungin.

4. The composition of claim 1, further comprising water.

5. The composition of claim 4, wherein said composition is in the form of a gel.

6. The composition of claim 1, wherein said composition is configured to form a gel in situ when ingested orally.

7. The composition of claim 1, wherein:
i) said methyl cellulose is present in said composition at about 1-3%,
ii) said HPMC comprises 80-120 cp HPMC or HPMC 615, and/or
iii) said PVP is present in said composition at about 0.5%-2.0%.

8. The composition of claim 7, wherein said carrier agent further comprises at least one ion-dependent polymer.

9. The composition of claim 1, wherein said composition is configured to be gastrointestinal retentive when taken orally.

10. A method of treating a subject with a fungal infection and/or inflammatory bowel disease comprising: administering the composition of any of claims 1-9 to the subject, or providing the composition of any of claims 1-9 to the subject such that they administer the composition to themselves.

11. The method of claim 10, wherein said administering or said administer the composition to themselves, comprises oral administration.

12. The method of claim 10, wherein said administering or said administer the composition to themselves, comprises intracolonic administration.

13. The method of claim 10, wherein said subject has a fungal infection selected from: candidemia, esophageal candidiasis, and aspergillosis.

14. The method of claim 10, wherein said subject has Crohn's disease.

15. The method of claim 10, wherein said administering or said administer the composition to themselves, comprises oral administration; and wherein said echinocandin exerts its fungicide effect locally in the gastrointestinal tract.

16. The method of claim 15, wherein said composition is mucoadhesive.

17. The method of claim 10, wherein said subject is a human.
